(19) United States
(12) Patent Application Publication
(10) Pub. No.: US 2009/0197957 A1
(43) Pub. Date: Aug. 6, 2009

(54) Methods and compositions for the treatment of pain

(75) Inventors: Michael Lionel Selley, New South Wales (AU); Richard Owen Williams, London (GB); Julia Jane Inglis, England (GB)

Correspondence Address: WILSON SONSINI GOODRICH & ROSATI, 650 PAGE MILL ROAD, PALO ALTO, CA, 94304-1050, US

(73) Assignee: Nuon Therapeutics, Inc.

(21) Appl. No.: 11/914,442

(22) PCT Filed: May 16, 2006
(86) PCT No.: PCT/AU06/00649

§ 371 Date: Jun. 23, 2008

Related U.S. Application Data
(60) Provisional application No. 60/681,323, filed on May 16, 2005.

(30) Foreign Application Priority Data
Feb. 21, 2006 (AU) 2006900855

(51) Int. Cl. A61K 31/196; C07C 229/48; A61P 31/18

(52) U.S. Cl. 514/563; 562/458

(57) ABSTRACT

The present invention relates generally to the area of pain management and more particularly, to a method of analgesia and agents useful for same. More particularly, the present invention relates to a method of analgesia utilising a compound of formula (I). The method of the present invention is useful, inter alia, in the therapeutic or prophylactic treatment of pain, including acute pain, chronic non-malignant pain and chronic malignant pain. Also provided are compounds for use in the method of the invention.

### FIELD OF THE INVENTION

The present invention relates generally to the area of pain management and more particularly, to a method of analgesia and agents useful for same. More particularly, the present invention relates to a method of analgesia utilising a compound of formula (I). The method of the present invention is useful, inter alia, in the therapeutic or prophylactic treatment of pain, including acute pain, chronic non-malignant pain and chronic malignant pain. Also provided are compounds for use in the method of the invention.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Pain is inadequately treated in many situations. In particular, pain is thought to be inadequately treated in half of all surgical procedures. In addition to immediate unpleasantness, painful experiences can imprint themselves indelibly on the nervous system, amplifying the response to subsequent noxious stimuli (hyperalgesia) and causing typically painless sensations to be experienced as pain (allodynia). A chronic condition sometimes develops that produces continuous pain long after surgery. Prior painful experiences are a known predictor of increased pain and analgesic use in subsequent events or experiences.

Both the peripheral and the central nervous system (CNS) are involved in the perception of pain, with the spinal and supraspinal components of the CNS playing key roles (Fields H. L., Pain, New York: McGraw-Hill, 1987). The transduction of noxious stimuli begins with peripheral nociceptors. Signals from these nociceptors travel primarily along small myelinated A and unmyelinated C fibers with soma lying in the dorsal root ganglion. The axons synapse in the dorsal horn of the spinal cord, where the neurons of laminae I, II and V are most involved in the perception of pain.

The signals then travel along the spinothalamic tract of the spinal cord to the thalamus and the cortex, Large fiber inputs from other sensory modalities and descending pathways can modulate activity in the dorsal horn, where these descending pathways may provide a physiologic explanation for the increased pain experienced by patients who have high levels of depression and anxiety (Taenzer et al., Pain, 24:331-42, 1986; Haythornthwaite et al., J. Urol., 160:1761-4, 1998). Painful stimuli ultimately cause activity in both the somatotopically appropriate portion of the sensory cortex and the limbic system (Rainville et al., Science 277:968-71, 1997).

The response to noxious stimuli can be modulated by their repeated application (Fields 1987, supra). For example, peripheral nociceptors become more responsive with the repeated application of noxious stimuli. Their sensitivity can be further enhanced by many tissue factors and inflammatory mediators released in the course of tissue injury. The response of neurons in the dorsal horn of the spinal cord of experimental animals has been found to be biphasic. The initial response to a noxious stimulus is brief and correlates with the sharp, well-localized initial pain. The second phase of the response is more prolonged and correlates with the dull, diffuse pain experienced after the initial injury. Experimentally, this second phase is associated with a growing region of hypersensitivity around the point where the noxious stimulus was initially applied.

The process through which the neurons of the dorsal horn of the spinal cord become sensitized by prior noxious stimuli is often referred to as “windup” or “central sensitization”. Much less is known about pain-induced sensitization of the supraspinal components of the CNS. Collectively, however, the above mechanisms enhance sensitivity to noxious stimuli and may increase the level of pain experienced following surgery.

Pain can be classified as acute, chronic non-malignant, or chronic malignant. Headaches, the most common cause of pain, can be considered a separate class of pain. Acute pain usually is due to mechanical or thermal (usually heat) injuries. Examples of mechanical injuries include surgery, soreness of muscles due to overuse or strain, tears of the ligaments, broken bones, bruises, and cuts. Chronic non-malignant pain is a type of pain associated with progressive, debilitating diseases such as arthritis. Chronic malignant pain is pain associated with advanced, progressive disease (often terminal) such as cancer, multiple scleroses, AIDS and terminal kidney disease.

In light of the fact that there is a diversity in classes of pain, and in particular the fact that in many situations pain is not adequately treatable, there is an ongoing need to identify and develop new methods of treating pain—whether that be therapeutically (after the onset of pain) or prophylactically (preemptively).

In work leading up to the present invention it has been very surprisingly determined that tranilast can function as an analgesic. This finding is of great significance since it now provides another option in terms of the treatment of a symptom which some patients will attest can be worse to live with than the disease itself.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method for inducing analgesia in a subject, said method comprising administering to said subject an effective amount of a compound of formula (I):

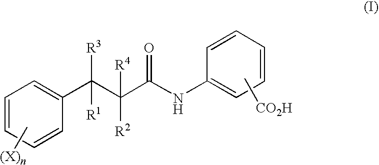

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

Another aspect of the present invention provides a method for inducing analgesia in a subject, said method comprising administering to said subject an effective amount of tranilast.

In yet another aspect there is provided a method for prophylactically inducing analgesia in a subject, said method comprising administering to said subject an effective amount of a compound of formula (I):

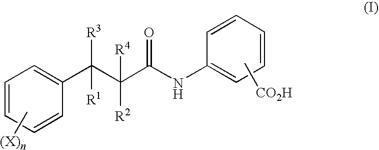

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

In still another aspect there is provided a method for inducing analgesia in a mammal, said method comprising administering to said mammal an effective amount of a compound of formula (I):

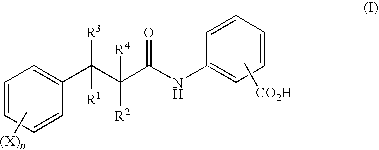

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

Yet still another aspect of the present invention is directed to a method of downregulating analgesia in a subject, said method comprising administering to said subject an antagonist of a compound of formula (I) or a pharmaceutically acceptable salt thereof.

Still yet another aspect of the present invention is directed to a method for the treatment and/or prophylaxis of pain in a subject, said method comprising administering to said subject an effective amount of a compound of formula (I):

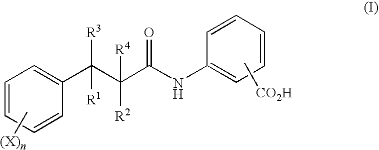

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

In a related aspect, there is provided a method for the treatment and/or prophylaxis of a condition in a subject, which condition is characterised by symptoms of pain, said method comprising administering to said subject an effective amount of a compound of formula (I):

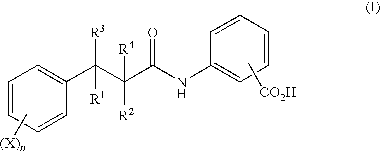

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, for a time and under conditions sufficient to inhibit or reduce said pain.

Another aspect of the present invention relates to the use of a compound of formula (I):

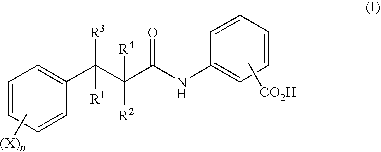

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, in the manufacture of a medicament for the treatment of a condition in a mammal, which condition is characterised by pain, wherein said compound of formula (I) induces analgesia.

Yet another aspect of the present invention relates to compounds of formula (I) or pharmaceutically acceptable salts thereof or antagonists thereof, as hereinbefore defined, when used in the method of the present invention.

### BRIEF DESCRIPTION OF THE DRAWINGS

FIG. 1 is a graphical representation of the impact of tranilast on reducing mechanical allodynia following collagen-induced arthritis.

FIG. 2 is a graphical representation of the treatment of established CIA with 3,4-DAA. DBA/1 mice were immunised with type II collagen in CFA and monitored for development of arthritis. On day 1 of arthritis, mice were injected intraperitoneally with 3,4-DAA on a daily basis. Paw thickness was measured with callipers. The clinical scoring system was as follows: 0=normal, 1=slight swelling and/or erythema, and 2=pronounced oedematous swelling. Each limb was graded, giving a maximum score of 8 per mouse. Histological assessment of arthritis was carried out on haematoxylin and eosin stained sections using a scoring system as follows: 0, normal; 1, minimal synovitis without cartilage/bone erosion; 2, synovitis with some marginal erosion but joint architecture maintained; 3, severe synovitis and erosion with loss of normal joint architecture. There were 14 mice/group (data pooled from two separate experiments).

*, P<0.05 (compared to control group).

FIG. 3 is a graphical representation depicting that treatment with 3,4-DAA leads to increased IL-10 levels in vivo. Mice with established CIA were treated with 3,4-DAA or vehicle (n=7) for 10 days (see FIG. 2), then bled. IL-10 in the sera was measured by ELISA.

FIG. 4 is a graphical representation of the results of mice with established CIA being treated for 10 days with 3,4-DAA or vehicle control. Mice were then killed and draining (inguinal) lymph node cells were cultured for 72 h in the absence or presence of type II collagen. IFN-γ and IL-5 production was measured by ELISA and was found to be significantly reduced in the mice given 3,4-DAA at 400 mg/kg. However, on re-stimulation with collagen, differences between the groups were not significant, indicating that the ability of the T cells to respond to antigenic stimulation returned to normal in the absence of the drug.

FIG. 5 is a graphical representation of the relapse of arthritis 4 days after cessation of therapy. Mice with established CIA (n=6) were treated with 3,4-DAA (400 mg/kg/day) from days 1 to 5 of arthritis and clinical severity of arthritis was monitored up to day 12. Arthritis is seen to relapse at around day 9.

FIG. 6 is an image depicting that 3,4-DAA inhibits mechanical and thermal allodynia, and inhibits astrocytic activation in arthritic mice. Mechanical (a) and thermal (b) allodynia, and paw swelling (c) and clinical score (d) were assessed in naïve mice on the day of arthritis onset, and up to 10 days following therapy with 200 mg/kg 3,4-DAA, 0.5 mg/2 days dexamethasone, or vehicle. 3,4-DAA abolished thermal (a) and mechanical (b) allodynia compared to controls, whilst dexamethasone significantly reduced thermal allodynia 3 days following onset only, and had no action on mechanical allodynia. In contrast both 3,4-DAA and dexamethasone reduced significantly reduced paw swelling (c) and clinical score (d) to a similar degree. (e) Immunohistochemistry on the lumbar spinal cord showed little GFAP expression in the naïve mouse (top right panel), and astrocyte hyperplasia and increased GFAP expression 10 days following CIA onset (top left panel). Whilst 3,4-DAA therapy reduced astrocytic activation (bottom right panel) dexamethasone had no action on GFAP levels (bottom left panel). Quantification of the number of hyper-plastic astrocytes in the spinal cord 10 days following onset of arthritis was performed (f). CIA induced a 5-fold increase in the number of activated astrocytes, which was significantly reduced by 3,4-DAA therapy, but not affected by dexamethasone therapy.

FIG. 7 is a graphical representation depicting that 3,4-DAA and 3-HAA inhibit B and T cell proliferation in vitro. Purified B and T cells were stimulated for 72 h with anti-CD40 (a), or anti-CD3/anti-CD28 (b) respectively, in the presence of varying doses of 3,4-DAA, or 3-HAA. Both 3,4-DAA, and 3-HAA dose-dependently inhibited B and T cell proliferation, assessed by 3H-thymidine incorporation. Both 3,4-DAA and 3-HAA therapy dose-dependently reduced IFN-γ production by T-cells (c). 3,4-DAA dose-dependently inhibited IL-10 and IL-5 production (d, e), whilst 31-HAA increased IL-10 and IL-5 production by T-cells.

### DETAILED DESCRIPTION OF THE INVENTION

The present invention is predicated, in part, on the surprising determination that compounds of formula (I) exhibit analgesic properties. This finding has now facilitated the development of therapeutic and prophylactic means for treating pain, in particular in the context of treating the pain which is symptomatic of many disease conditions. Also provided are compositions for use in the present invention.

Accordingly, one aspect of the present invention is directed to a method for inducing analgesia in a subject, said method comprising administering to said subject an effective amount of a compound of formula (I):

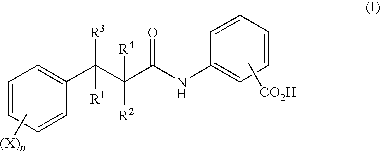

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

The carboxyl group may be in the 2-, 3- or 4-position of the aromatic ring. Preferably the carboxyl group is in the 2-position.

Preferably at least one of R¹ and R² is a hydrogen atom. More preferably, both of R¹ and R² are hydrogen atoms.

Preferably R³ and R⁴ taken together form a chemical bond. Such compounds having an unsaturated bond may be in the form of E or Z geometric isomers.

Preferably n is 1 or 2 and each X, which may be the same or different, is selected from halogen, C₁-C₄ alkyl or C₁-C₄alkoxy. Preferably X is selected from halogen and C₁-C₄alkoxy. More preferably, n is 2 and both X are selected from C₁-C₄alkoxy, especially when both X are methoxy.

Particularly preferred compounds useful in the invention are those of formula (II):

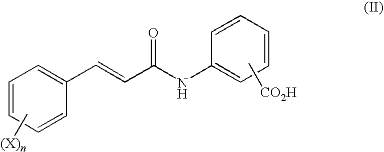

Examples of compounds of formula (II) include
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

A particularly preferred compound of formula (II) for use in the invention is 2-[[3-(3,4-dimethoxyphenyl)-1-oxo-2-propenyl]amino]benzoic acid (tranilast, TNL).

As used herein, the term “C₁-C₄alkyl” refers to linear or branched alkyl groups having 1 to 4 carbon atoms. Examples of such groups include methyl, ethyl, n-propyl, isopropyl, n-butyl, sec-butyl and tert-butyl.

As used herein, the term “C₁-C₄alkoxy” refers to hydroxy groups substituted with linear or branched alkyl groups having 1 to 4 carbon atoms. Examples of such groups include methoxy, ethoxy, n-propoxy, isopropoxy, n-butoxy, sec-butoxy and tert-butoxy.

As used herein, the term “halogen” or “halo” refers to fluoro, chloro or bromo atoms.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Compounds of formula (I) and their pharmaceutically acceptable salts are known and may be prepared by methods known in the art, see U.S. Pat. No. 3,940,422 the contents of which are incorporated herein by reference.

It will also be recognised that some compounds of formula (I) may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

Without limiting the present invention to any one theory or mode of action, the compounds of formula (I) are orally active anti-allergic compounds. A particularly preferred compound of the invention is known by either of the chemical names N-[3,4-dimethoxycinnamoyl]-anthranilic acid or 2-[[3-(3,4-dimethoxyphenyl)-1-oxo-2-propenyl]amino]benzoic acid and may also be referred to as Tranilast. Still further, it is known by the chemical formula C₁₈H₁₇NO₅ and by the trade name Rizaben. The structure of N-[3,4-dimethoxycinnamoyl]-anthranilic acid is depicted below:

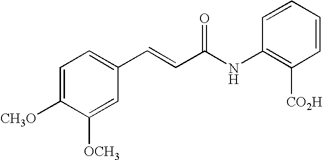

The present invention therefore preferably provides a method for inducing analgesia in a subject, said method comprising administering to said subject an effective amount of tranilast.

Reference to the terms “analgesia” and “analgesic response” is intended to describe a state of reduced sensitivity to pain, which preferably occurs without overt sedation and preferably without an effect upon the sense of touch. Preferably, the sensitivity to pain is reduced by at least 30%, preferably at least 50%, more preferably at least 70% and particularly preferably at least 85%. In a most preferred aspect of the present invention, the sensitivity to pain is completely, or substantially completely, removed. To assess the level of reduction of sensitivity to pain associated with the analgesia induced by the methods according to the present invention, it is possible to conduct tests such as the short form McGill pain questionnaire and/or visual analogue scales for pain intensity and/or verbal rating scales for pain intensity and/or measurement of tactile allodynia using von Frey hairs or similar device. These tests are standard tests within the art and would be well known to the skilled person.

By the phrase “overt sedation” it is intended to convey that the methods (and compositions) of the invention do not result in practically meaningful sedation of the patient or subject being treated, i.e. significant, visible or apparent drowsiness or unconsciousness of the patient being treated. Thus, the treatment methods of the invention do not result in sleepiness or drowsiness in the patient that interfere with, or inhibit, the activities associated with day to day living, such as driving a motor vehicle or operating machinery for human subjects, or feeding and grooming for animal subjects.

As detailed hereinbefore, it has been surprisingly determined that compounds of the formula (I), in particular tranilast, induce analgesia and are therefore useful in the treatment of pain. To this end, reference to “pain” should be understood as a reference to any form of pain, irrespective of its aetiology. Without limiting the present invention to any one theory or mode of action, the sensation of pain is generally the outcome or symptom of a process related to disease onset or progression or some other aberrant physiological event. Pain can be broadly classified as follows:

(i) Acute pain—associated with mechanical or thermal injuries;
(ii) Headache pain;
(iii) Chronic non-malignant pain—associated with progressive debilitating diseases;
(iv) Chronic malignant pain—associated with advanced, progressive diseases.

Reference to “pain” herein should be understood to encompass all these forms of pain. Preferably, said pain is the pain associated with an inflammatory condition, herein referred to as “inflammatory pain”. An example of inflammatory pain is the pain of rheumatoid arthritis inflammation or other autoimmune disorders such as systemic lupus erythematosus or osteoarthritis.

Reference to “inducing” analgesia should be understood as a reference to upregulating or otherwise causing the onset of analgesia. Accordingly, the method of the present invention may be utilised to augment or otherwise agonise on existing pain relief regime or it may induce analgesia where no analgesic has yet been administered. Still further, it should be understood that the subject analgesia may be induced either therapeutically or prophylactically. A therapeutic regime is one where tranilast is administered subsequently to the onset of pain in order to reduce or eliminate the pain sensation. A prophylactic regime, however, is where tranilast is administered prior to the onset of pain, that is, as a pre-emptive analgesic. This latter form of pain relief is of particular importance since it is now generally recognised that preventing breakthrough pain is more effective than treating pain after its onset. Without limiting the present invention to any one theory or mode of action, it is also thought that pre-emptive analgesia is highly desirable in the longer term since it is thought to reduce or even eliminate the hypersensitivity to a noxious stimuli which can occur when a patient is repeatedly subjected to a pain experience. Accordingly, preferably said analgesia is induced prophylactically.

According to this preferred embodiment there is provided a method for prophylactically inducing analgesia in a subject, said method comprising administering to said subject an effective amount of a compound of formula (I):

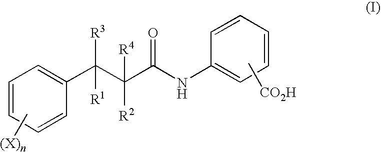

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

Preferably, said compound is tranilast.

The term “subject” as used herein includes reference to all mammalian and non-mammalian animals. Mammalian animals includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human. Reference to non-mammalian animals includes amphibians, fish, reptiles and birds.

The present invention therefore most preferably provides a method for inducing analgesia in a mammal, said method comprising administering to said mammal an effective amount of a compound of formula (I):

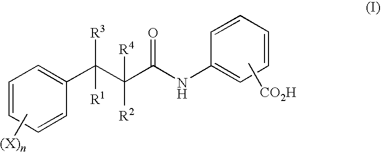

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

Preferably, said compound is tranilast.

Even more preferably, said tranilast is administered prophylactically.

Most preferably, said pain is inflammatory pain.

Although the preferred method is to induce analgesia, it may also be desired to partially or fully restore the sensation of pain in certain circumstances. For example, in the context of certain injuries, it may be desirable to initially alleviate pain by administering a compound of formula (I). However, when medical attention is subsequently sought, it may be necessary for the physician to examine the patient in the absence of pain relief such that the patient can provide information or guidance in relation to the nature or location of the pain. Accordingly, to the extent that it is not possible to rectify this situation by ceasing administration of compounds of formula (I), it may be desirable to administer, (in a site directed manner, for example) an antagonistic agent of compounds of formula (I). In another example, therapy with compounds of formula (I) may necessitate the use of antagonists of compounds of formula (I) in order to inhibit the functioning of the compound which has been introduced to a mammal but which functional activity is required to be slowed or stopped. Reference to an “antagonist of formula (I) functioning” should therefore be understood to mean that at least some of the analgesic effect which has been induced by said compound is inhibited, slowed or otherwise retarded due to the functional effects of the antagonist.

Accordingly, another aspect of the present invention is directed to a method of downregulating analgesia in a subject, said method comprising administering to said subject an antagonist of a compound of formula (I) or a pharmaceutically acceptable salt thereof.

Reference to “antagonist of a compound of formula (I) or a pharmaceutically acceptable salt thereof” should be understood as a reference to any proteinaceous or non-proteinaceous molecule which directly or indirectly inhibits, retards or otherwise downregulates the analgesic activity of the compounds of formula (I) or pharmaceutically acceptable salts thereof. Identification of antagonists suitable for use in the present invention can be routinely achieved utilising methods well known to those skilled in the art.

A further aspect of the present invention relates to the use of the invention in the context of the treatment and/or prophylaxis of pain, in particular in the context of treating the pain which is symptomatic of many disease conditions or other aberrant conditions.

Accordingly, another aspect of the present invention is directed to a method for the treatment and/or prophylaxis of pain in a subject, said method comprising administering to said subject an effective amount of a compound of formula (I):

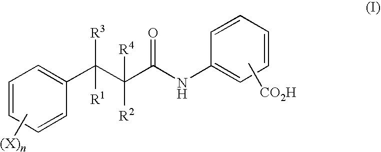

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

In a related aspect, there is provided a method for the treatment and/or prophylaxis of a condition in a subject, which condition is characterised by symptoms of pain, said method comprising administering to said subject an effective amount of a compound of formula (I):

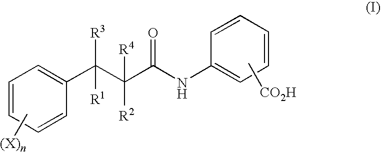

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, for a time and under conditions sufficient to inhibit or reduce said pain.

Preferably, said compound is tranilast.

More preferably, said subject is a mammal and most preferably a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Reference to a “condition characterised by symptoms of pain” should be understood as a reference to any disease or non-disease condition which is associated either with ongoing chronic pain or one or more episodes of transient pain, such as an episode of acute pain. The subject condition may be a disease condition, such as cancer, infection, inflammation, autoimmune conditions, AIDS, kidney disease or multiple sclerosis. However, it may also correspond to a non-disease condition which is nevertheless associated with pain, such as a post-operative surgical condition or even a physiologically normal condition or response which is nevertheless associated with pain such as the pain associated with menstruation or childbirth or the headaches which are sometimes referred from tense shoulder muscles. Preferably, said condition is an inflammatory condition and more particularly an autoimmune condition such as rheumatoid arthritis, systemic lupus erythematosus or osteoarthritis.

The present invention further contemplates a combination of therapies, such as the administration of compounds of formula (I) or pharmaceutically acceptable salts thereof together with subjection of the mammal to other agents which are useful in the treatment of the subject condition. For example, one might administer the pain relief of the present invention together with a treatment directed to ameliorating the cause of the disease, such as chemotherapy or radiotherapy in the context of cancer. Where the subject pain is the result of a condition caused by an infection, there may be co-administered anti-viral, anti-parasitic or antibiotic agents.

Administration of the compounds of formula (I) or pharmaceutically acceptable salts thereof or antagonist thereof (herein referred to as “modulatory agent”), in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (eg. using slow release molecules). The modulatory agent may be administered in the form of pharmaceutically acceptable nontoxic salts, such as acid addition salts or metal complexes, eg. with zinc, iron or the like (which are considered as salts for purposes of this application). Illustrative of such acid addition salts are hydrochloride, hydrobromide, sulphate, phosphate, maleate, acetate, citrate, benzoate, succinate, maleate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

The modulatory agent may be linked, bound or otherwise associated with any proteinaceous or non-proteinaceous molecules. For example, in one embodiment of the present invention said modulatory agent may be associated with a molecule which permits targeting to a localised region.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of a compound of formula (I):

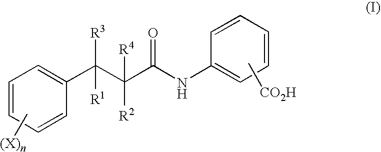

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, in the manufacture of a medicament for the treatment of a condition in a mammal, which condition is characterised by pain, wherein said compound of formula (I) induces analgesia.

The present invention contemplates the administration of the compounds of formula (I) either alone or as a pharmaceutical composition comprising a compound of formula (I) or a pharmaceutically acceptable salt thereof or antagonist thereof as hereinbefore defined and one or more pharmaceutically acceptable carriers and/or diluents. Said agents are referred to as the active ingredients.

The present invention also relates to compositions comprising the modulatory agent, optionally with another analgesic agent, together with one or more pharmaceutically acceptable additives and optionally other medicaments, as detailed above. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compositions suitable for topical administration to the skin, i.e. transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds of the present invention may also be presented as implants, which may comprise a drug bearing polymeric device wherein the polymer is biocompatible and non-toxic. Suitable polymers may include hydrogels, silicones, polyethylenes and biodegradable polymers.

The compounds of the subject invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms. For slow release administration, the active ingredients may be suspended as slow release particles or within liposomes, for example.

The pharmaceutical compositions of the present invention may be packaged for sale with other active agents or medicaments as hereinbefore described.

Yet another aspect of the present invention relates to compounds of formula (I) or pharmaceutically acceptable salts thereof or antagonists thereof, as hereinbefore defined, when used in the method of the present invention.

### Example 1

### Evaluation of the Analgesic Activity of Tranilast

### Summary

Tranilast was evaluated for possible analgesic activity in the mouse acetic acid-induced writhing model. Tranilast at 100, 200 and 400 mg/kg was administered orally (PO) 1 hour before intraperitoneal injection of acetic acid (0.5%, 20 ml/kg). Tranilast at 100, 200 and 400 mg/kg appeared to cause dose-dependent inhibition of acetic acid-induced writing in mice; tranilast at 200 and 400 mg/kg was associated with 24% and 47% inhibition of writhing response, respectively, relative to vehicle control when administered 1 hour before acetic acid injection.

### Materials and Equipment

### Test Substances and Dosing Pattern

Tranilast, provided by Angiogen Pharmaceuticals Pty. Ltd., was dissolved in 1% NaHCO₃ (heated to 70° C.) and administered orally at doses of 100, 200 and 400 mg/kg at 60 minutes before acetic acid injection. The dosing volume was 10 ml/kg.

### Animals

Male CD-1 (Crl.) derived mice weighing 24±2 g were provided by BioLasco Taiwan (under a Charles River Laboratories Technology licensee). Space allocation for 10 animals was 29×18×13 cm. Mice were housed in APEC^{R} cages. All animals were maintained in a controlled temperature (22° C.-24° C.) and humidity (60%-70%) environment with 12 hours light dark cycles for at least one week in MDS Pharma Services—Taiwan Laboratory prior to use. Free access to standard lab chow for mice (Lab Diet, Rodent Diet, PMI Nutrition International, USA) and tap water was granted. All aspects of this work including housing, experimentation and disposal of animals were performed in general accordance with the Guide for the Care and Use of Laboratory Animals (National Academy Press, Washington, D.C., 1996).

### Chemicals

Acetic Acid (Sigma, USA), Ibuprofen (Sigma, USA) and NaHCO₃ (Merck, Germany).

### Equipment

Animal case (ShinTeh, R.O.C.), Beaker 1000 ml (Kinmax, USA), Hypodermic needle 25G x 1″ (Top Corporation, Japan), Mouse scale X-40 (Taconic, USA), Needle for oral administration (Natsume, Japan), Syringe 1 ml (Top Corporation, Japan) and Stop watch (World Leader, Swiss).

### Method

### Analgesia, Acetic Acid Writhing.

Test substance was administered PO (400, 200 and 100 mg/kg) to groups of 5 CD-1 (Crl.) derived male or female mice weighing 22±2 g one hour before injection of acetic acid (0.5%, 20 ml/kg IP). Reduction in the number of writhes by 50 percent or more (≧50%) per group of animals observed during the 5 to 10 minute period after acetic acid administration, relative to the vehicle-treated control group, indicates possible analgesic activity.

### Table of Results

TABLE 1
Assay #503900 Analgesia, Acetic Writhing in Mice

| | | | | | No. of Writhing | | % |
|---|---|---|---|---|---|---|---|
| Treatment | Route | Dose | N | B.W. | Individual | Average | Inhibition |
| Vehicle | PO | 10 ml/kg | 1 | 22 | 19 | | |
| (1% NaHCO₃) | | | 2 | 22 | 20 | | |
| | | | 3 | 22 | 15 | | |
| | | | 4 | 23 | 16 | | |
| | | | 5 | 22 | 17 | 17 | — |
| PT # 104283-ADD | PO | 400 mg/kg | 1 | 22 | 3 | | |
| (AGG-1) (Tranilast) | | | 2 | 22 | 16 | | |
| | | | 3 | 23 | 2 | | |
| | | | 4 | 23 | 10 | | |
| | | | 5 | 23 | 14 | 9 | 47 |
| | PO | 200 mg/kg | 1 | 22 | 10 | | |
| | | | 2 | 22 | 19 | | |
| | | | 3 | 22 | 9 | | |
| | | | 4 | 22 | 10 | | |
| | | | 5 | 22 | 16 | 13 | 24 |
| | PO | 100 mg/kg | 1 | 22 | 19 | | |
| | | | 2 | 22 | 15 | | |
| | | | 3 | 22 | 16 | | |
| | | | 4 | 22 | 17 | | |
| | | | 5 | 22 | 9 | 15 | 12 |
| Ibuprofen | PO | 30 mg/kg | 1 | 22 | 6 | | |
| | | | 2 | 22 | 8 | | |
| | | | 3 | 22 | 10 | | |
| | | | 4 | 22 | 10 | | |
| | | | 5 | 22 | 8 | 8 | (53) |

Test substance was administered orally to groups of 5 mice 60 minutes before injection of Acetic Acid (0.5%, 20 ml/kg IP). The number of writhing per group of animals observed during the 5 to 10 minutes period after acetic acid challenge was observed. Reduction in the number of writhings by 50 percent or more (≧50%) relative to the vehicle-treated control group indicates possible analgesic activity.

### Example 2

### The Effect of Tranilast on Cyclo-Oxygenase-2

The U.S. Food and Drug administration (FDA) has decided that the widely used cyclo-oxygenase-2 (COX-2) inhibitors rofecoxib (Vioxx®) and celecoxib (Celebrex®) should carry black box warnings because they both carry a serious risk of heart attack or stroke (www.fda.gov/cder; Lenzer, B.M.J. 2005; 330:440). The FDA has also ordered the withdrawal of valdecoxib (Bextra®) from the market (www.fda.gov/cder). These actions were taken following the voluntary withdrawal of rofecoxib after it was found to double the risk of heart attacks or strokes in patients in a colorectal adenoma prevention trial (Bresalier et al., N. Engl. J. Med. 2005; 352:1092-1102). In a similar trial with celecoxib there was triple the risk of adverse cardiovascular events (Solomon et al., N. Engl. J. Med. 2005; 352:1071-1080). The short-term use of valdecoxib and its intravenous prodrug, parecoxib, caused an increased incidence of cardiovascular events following coronary artery by-pass surgery (Nussmeier et al., N. Engl. J. Med. 2005; 352:1081-1091). The association of three structurally diverse COX-2 inhibitors with cardiovascular complications suggests a class effect (Drazen N. Engl. J. Med. 2005; 352:1131-1132). It is believed these drugs inhibit endothelial COX-2, leading to the suppression of endothelial prostaglandin I₂, increasing blood pressure, accelerating atherosclerosis and exaggerating the thrombotic response to the rupture of atherosclerotic plaques (FitzGerald, N. Engl. J. Med. 2004; 351:1709-1711). The FDA has concluded that an increased risk of cardiovascular events may also be a class effect of non-selective non-steroidal anti-inflammatory drugs (NSAIDs) that inhibit both COX-1 and COX-2 and is requesting a black box warning on all prescription non-selective NSAIDs (www.fda.gov/cder).

The mechanism of action of tranilast as an analgesic is unknown. However, it has been shown to have no effect on the activity of either the COX-1 or the COX-2 enzymes (data attached). Moreover, there was no significant increase in adverse cardiovascular events in a clinical trial of tranilast in 11,484 patients with restenosis following percutaneous coronary intervention (Holmes et al., Circulation 2002; 106:1243-1250). The data suggest that tranilast may be an effective analgesic agent without the adverse cardiovascular effects of COX-2 inhibitors and non-selective NSAIDs.

### Methods

Methods employed in this study have been adapted from the scientific literature to maximize reliability and reproducibility. Reference standards were run as an integral part of each assay to ensure the validity of the results obtained. Assays were performed under conditions described in the accompanying “Methods” section of this report. The literature references for each assay are in the “Literature References” section. If either of these sections were not originally requested with the accompanying report, please contact us at the number below for a printout of either of these report sections.

116020 Cyclooxygenase COX-1

| | |
|---|---|
| Source: | Human platelets |
| Substrate: | 100 μM Arachidonic Acid |
| Vehicle: | 1% DMSO |
| Pre-Incubation Time/Temp: | 15 minutes @ 37° C. |
| Incubation Time/Temp: | 15 minutes @ 37° C. |
| Incubation Buffer: | HBSS with 15 Mus musculus HEPES, |
| | pH 7.4 |
| Quantitation Method: | EIA quantitation of PGE₂ |
| Significance Criteria: | ≧50% of max stimulation or |
| | inhibition |

118010 Cyclooxygenase COX-2

| | |
|---|---|
| Source: | Human recombinant insect Sf21 |
| Substrate: | 0.3 μM Arachidonic Acid |
| Vehicle: | 1% DMSO |
| Pre-Incubation Time/Temp: | 15 minutes @ 37° C. |
| Incubation Time/Temp: | 5 minutes @ 37° C. |
| Incubation Buffer: | 100 Mus musculus Tris-HCl, pH 7.7, |
| | 1 Mus musculus Glutathione, 1 μM |
| | Hematin, 500 μM Phenol |
| Quantitation Method: | EIA quantitation of PGE₂ |
| Significance Criteria: | ≧50% of max stimulation or |
| | inhibition |

Reference Compound Data - Biochemical Assays

| | | REFERENCE | HISTORICAL | | | CONCURRENT MIC | |
|---|---|---|---|---|---|---|---|
| CAT. # | ASSAY NAME | COMPOUND | IC₅₀ | K_{I} | n_{H} | BATCH* | IC₅₀ |
| 116020 | Cyclooxygenase | Indomethacin | 0.044 μM | | | 130743 | 0.0432 μM |
| | COX-1 | | | | | | |
| 118010 | Cyclooxygenase | Rofecoxib | 0.17 μM | | | 131083 | 0.162 μM |
| | COX-2 | | | | | | |

### Results

A summary of results meeting the significance criteria is presented in the following sections.

Biochemical assay results are presented as the percent inhibition of specific binding or activity throughout the report. All other results are expressed in terms of that assay's quantitation method (see Methods section).
For primary assays, only the lowest concentration with a significant response judged by the assays' criteria, is shown in this summary.
Where applicable, either the secondary assay results with the lowest dose/concentration meeting the significance criteria or, if inactive, the highest dose/concentration that did not meet the significance criteria is shown.
Unless otherwise requested, primary screening in duplicate with quantitative data (e.g., IC50±SEM, KI±SEM and nH) are shown where applicable for individual requested assays. In screening packages, primary screening in duplicate with semi-quantitative data (e.g., estimated IC50, Ki and nH) are shown where applicable (concentration range of 4 log units); available secondary functional assays are carried out (30 μM) and MEC or MIC determined only if active in primary assays >50% at 1 log unit below initial test concentration.
Please see Experimental Results section for details of all responses.

Significant responses (≧50% inhibition or stimulation for Biochemical assays) were noted in the primary assays listed below:

### Primary Tests

No significant responses noted.

TABLE 2
Biochemical Assay

| | | | | | | | †% Inhibition | | | | | | | | |
|---|---|---|---|---|---|---|---|---|---|---|---|---|---|---|---|
| | | Tranilast | | | | | −100 | −50 | 0 | 50 | 100 | | | | |
| Cat. # | Target | Batch | SPP. | n= | Conc. | % | ↓ | ↓ | ↓ | ↓ | ↓ | IC₅₀ | H_{I} | n_{H} | R |
| 116020 | Cyclooxygenase | 130743 | hum | 2 | 10 μM | −2 | | | | | | | | | |
| | COX-1 | | | | | | | | | | | | | | |
| 118010 | Cyclooxygenase | 131083 | hum | 2 | 10 μM | 0 | | | | | | | | | |
| | COX-2 | | | | | | | | | | | | | | |

* Batch: Represents compounds tested concurrently in the same assay(s).
‡ Partially soluble in in vitro test solvent.
Results with ≧50% stimulation or inhibition are highlighted. (Negative values correspond to stimulation of binding or enzyme activity).
R = Additional Comments
hum = human

### SUMMARY/CONCLUSION

None of the results met significance criteria at concentrations and/or doses used.

### Example 3

### Analgesic Activity of 3-Hydroxyanthranilic Acid

### Summary

3-Hydroxyanthranilic acid was evaluated for possible analgesic activity in mouse acetic acid-induced pain response assay. 3-Hydroxyanthranilic acid at doses of 400, 200 and 100 mg/kg PO did not demonstrate any significant analgesic activity (≧50% inhibition of writhing relative to the vehicle-treated control group); only at 400 mg/kg PO was associated with a moderate but non-significant 38% inhibition.

### Materials and Equipment

### Test Substances and Dosing Pattern

3-Hydroxyanthranilic acid was purchased from Sigma (USA) by MDS Pharma Services-Taiwan Ltd. and administered orally at the doses of 400, 200 and 100 mg/kg for analgesia, acetic acid-induced writhing assay. 2% Tween 80 was used as the vehicle.

### Animals

Male CD-1 (Crl.) mice provided by BioLasco Taiwan (under Charles River Laboratories Technology Licensee) were used. Space allocation for 10 animals was 29×18×13 cm. All animals were maintained in a controlled temperature (23° C.-24° C.) and humidity (60%-70%) environment with 12 hours light dark cycles for at least one week in MDS Pharma Services—Taiwan Laboratory prior to use. Free access to standard lab chow for Mice (LabDiet Rodent Diet, PMI Nutrition International, USA) and tap water was granted. All aspects of this work including housing, experimentation and disposal of animals were performed in general accordance with the Guide for the Care and Use of Laboratory Animals (National Academy Press, Washington, D.C., 1996).

### Chemicals

Acetic Acid (Sigma, USA), Tween 80 (Wako, Japan) and Aspirin (Sigma, USA).

### Equipment

Animal case (ShinTeh, R.O.C.), Beaker 1000 ml (Kinmax, USA), Hypodermic needle 25G x 1″ (Top Corporation, Japan), Mouse scale Z-40 (Taconic, USA), Needle for oral administration (Natsume, Japan), Syringe 1 ml (Top Corporation, Japan) and Stop watch (World Leader, Swiss).

### Method

### Analgesia, Acetic Acid Writhing

Test substance was administered orally to groups of 5 CD-1 (Crl.) derived male or female mice weighing 24±2 g, 2 hour before injection of acetic acid (0.5%, 20 ml/kg IP). Reduction in the number of writhes by 50 percent or more (≧50%) per group of animals observed during the 5 to 10 minute period after acetic acid administration, relative to a vehicle-treated control group, indicates possible analgesic activity.

### Table of Results

TABLE 3
Analgesia, Acetic Acid Writhing in Mice

| | | | | | No. of Writhing | | % |
|---|---|---|---|---|---|---|---|
| Treatment | Route | Dose | N | B.W. | Individual | Average | Inhibition |
| Vehicle | PO | 10 ml/kg | 1 | 24 | 14 | | |
| (2% Tween 80) | | | 2 | 24 | 14 | | |
| | | | 3 | 24 | 10 | | |
| | | | 4 | 25 | 14 | | |
| | | | 5 | 26 | 15 | 13 | — |
| PT # 1058283 | PO | 400 mg/kg | 1 | 25 | 5 | | |
| (AGG-2) | | | 2 | 25 | 6 | | |
| (3-Hydroxyanthranilic acid) | | | 3 | 25 | 14 | | |
| | | | 4 | 24 | 5 | | |
| | | | 5 | 24 | 12 | 8 | 38 |
| | PO | 200 mg/kg | 1 | 24 | 16 | | |
| | | | 2 | 26 | 9 | | |
| | | | 3 | 25 | 12 | | |
| | | | 4 | 25 | 10 | | |
| | | | 5 | 25 | 15 | 12 | 8 |
| | PO | 100 mg/kg | 1 | 25 | 18 | | |
| | | | 2 | 25 | 12 | | |
| | | | 3 | 24 | 13 | | |
| | | | 4 | 26 | 10 | | |
| | | | 5 | 25 | 12 | 13 | 0 |
| Aspirin | PO | 100 mg/kg | 1 | 24 | 0 | | |
| | | | 2 | 25 | 8 | | |
| | | | 3 | 25 | 3 | | |
| | | | 4 | 25 | 7 | | |
| | | | 5 | 26 | 9 | 5 | (62) |

Test substance was administered orally to groups of 5 mice 1 hour before injection of 0.5% Acetic Acid (20 ml/kg IP). The number of writhing per group of animals observed during the 5 to 10 minute period after acetic acid challenge was observed. Reduction in the number of writhing by 50 percent or more (≧50%) relative to the vehicle-treated control group indicates possible analgesic activity.

### Example 4

### The Analgesic Properties of Tranilast in Arthritis

### Materials and Methods

### Reagents

Type II collagen was purified from bovine cartilage, as described [Williams 2004, Methods Mol. Med. 98:207-216] and solubilized by stirring overnight at 4° C. in acetic acid (0.1M) or Tris buffer (0.05 M Tris, containing 0.2 M NaCl, pH 7.4). 3,4-DAA was synthesised by Angiogen Pharmaceuticals Pty. Ltd. For in vivo studies 3,4-DAA was dissolved at a maximum concentration of 10 mg/ml in 1% sodium bicarbonate by heating for 1 h at 70° C. Upon cooling, an emulsion was formed. For in vitro studies 3,4-DAA was dissolved in dimethyl sulphoxide (DMSO). 3-Hydroxy-anthranilic acid (3-HAA) was purchased from Sigma (Poole, UK) and dissolved in PBS.

### Induction and Assessment of Arthritis

Male DBA/1 mice (8-12 weeks old) were immunized intradermally at the base of the tail with bovine type II collagen (200 μg) emulsified in complete Freund's adjuvant (CFA; Difco, West Molesley, UK). Arthritis was monitored clinically using the following scoring system: 0=normal, 1=slight swelling and/or erythema, and 2=pronounced oedematous swelling. Each limb was graded, giving a maximum score of 8 per mouse. In addition, paw-swelling was measured using callipers.

Histopathological assessment of arthritis was carried out in a ‘blinded’ fashion on decalcified haematoxylin and eosin stained sections using a scoring system as follows: 0, normal; 1, minimal synovitis without cartilage/bone erosion; 2, synovitis with some marginal erosion but joint architecture maintained; 3, severe synovitis and erosion with loss of normal joint architecture. This research was approved by the local ethical review process committee and by the Home Office of Great Britain.

### Serum Anti-Collagen Antibody Levels.

ELISA plates (Nunc, Uxbridge, UK) were coated with 2 μg/ml of bovine CII dissolved overnight in Tris Buffer (0.05 M Tris, containing 0.2 M NaCl, pH 7.4) blocked with 2% bovine serum albumin (BSA) and then incubated with serial dilutions of test sera. A reference sample was included on each plate. Bound total IgG, IgG1 or IgG2a was detected by incubation with HRP-conjugated sheep anti-mouse IgG, IgG1 or IgG2a, followed by TMB substrate. Optical density was measured at 450 nm.

### Analysis of Lymph Node Cell Responses

Inguinal lymph nodes were excised from 3,4-DAA-treated and control mice. Alternatively, inguinal lymph nodes were removed from untreated arthritic mice (day 1-5 of arthritis) and 3,4-DAA was added in vitro. In both cases, a single cell suspension was prepared and LNC were cultured in RPMI 1640 containing FCS (10% v/v), 2-mercaptoethanol (20 μM), L-glutamine (1% w/v), penicillin (100 U/ml) and streptomycin (100 μg/ml) in the presence or absence of type II collagen (50 μg/ml). Secreted cytokines (IFN-γ, IL-5, and IL-10) were measured after 72 h. by ELISA. In brief, 96 well ELISA plates were coated with the respective capture antibody, blocked with bovine serum albumin (2% w/v), and then incubated with LNC culture supernatants overnight at 4° C. After washing, bound cytokines were detected using biotinylated detect antibodies. A standard curve was generated using known concentrations of the appropriate recombinant cytokine and the concentrations of cytokines present in culture supernatants were estimated by reference to the standard curve.

### B and T Cell Purification and Activation

A single cell suspension was prepared by pushing splenic tissue through a cell strainer, and erythrocytes were lysed using an ammonium chloride solution (Sigma, St Louis, Mo.). B cells were positively enriched by using anti-IgM microbeads (BD Pharmingen), and T cells were positively enriched using anti-CD4 MACS microbeads, according to the manufacturer's guidelines (Miltenyi Biotec, Bergisch Gladbach, Germany). Purity was assessed by flow cytometric analysis (B cell>90% CD19+, T cell>90% CD4+). Cells were cultured at 5×10⁵ cells/ml in 200 μl complete RPMI, as above, in a flat bottom 96-well plate and cultured for 72 h. B cells were stimulated with anti-CD40 monoclonal antibody (10 μg/ml; BD), and T cells were stimulated with 5 μg/ml plate-bound anti-CD3 (ebiosciences) plus 5 μg/ml soluble anti-CD28 (ebiosciences). 3,4-DAA, 3-HAA, or vehicle (DMSO) was added at graded concentrations immediately prior to stimulation. 48 hours after stimulation, 100 μl culture medium was collected, and cells were pulsed with 1 μCi ³H thymidine per well for 18 h. Cells were then harvested and plates assessed for thymidine incorporation. Each assay was performed on a minimum of 3 occasions. IFN-γ, IL-10 and IL-5 levels were assessed in the culture medium by ELISA, as above.

Allodynia Assessment with 3,4-DAA Therapy

The pain thresholds of the mice were assessed prior to immunization (naïve) on day of onset (day 0) and up to 10 days following therapy with 3,4-DAA (200 mg/kg/day), dexamethasone (0.5 mg/kg/2 days) or vehicle alone (n=9 per group). The Ugo Basile 37400 Plantar Von-Frey microprocessor controlled unit was used to assess mechanical hyperalgesia and Ugo Basile 7370-6 Plantar Test (Hargreaves test) was used to assess thermal hyperalgesia. Mechanical hyperalgesia was assessed by applying an increasing force to the hind paw at the rate of 3 g/second, and measuring the force required to elicit lifting of the paw. Thermal hyperalgesia was assessed by applying an increasing infrared source (intensity 50), and measuring the time required for lifting of the paw.

### Immunohistochemistry

Upon completion of treatment, animals were sacrificed by CO₂ exposure, and the lumbar spinal cord was excised, fixed (10% formalin), and embedded in paraffin. Immunohistochemistry was then performed to detect astrocytes with a rabbit-anti GFAP (glial fibrillary acidic protein) antibody (Dako Cytomation, Glostrup, Denmark). Antibody detection was performed using an ABC peroxidase method (Vector Laboratories, High Wycombe, Bucks., U.K.) (32).

### Statistical Analysis

Group means were analysed by one-way analysis of variance, followed by the Dunnett Multiple Comparisons test, where appropriate.

### Results

3,4-DAA inhibits the development of CIA

In order to assess its anti-arthritic potential, 3,4-DAA was injected into DBA/1 mice (200 mg/kg/day) from the day of immunisation with type II collagen in CFA. By day 28, 5 of 7 (71%) vehicle treated mice had developed arthritis of moderate severity (clinical score 2.8±0.6), whilst 1 of 7 (14%) 3,4-DAA-treated mice had developed mild arthritis (clinical score 1). Analysis of the sera of treated and control mice revealed no change in anti-collagen IgG1 or IgG2a levels in 3,4-DAA-treated mice.

### 3,4-DAA Reduces the Severity of Established Arthritis

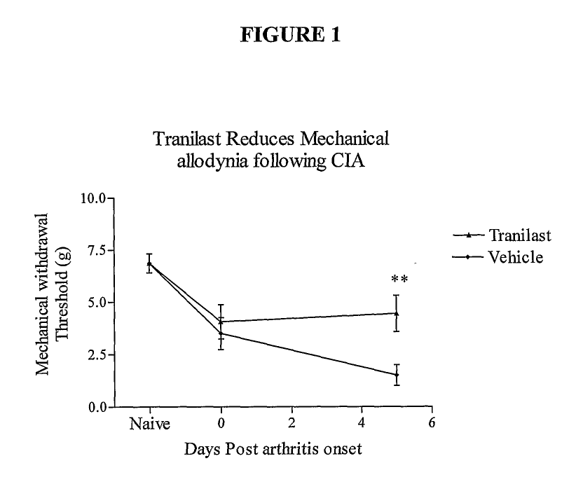

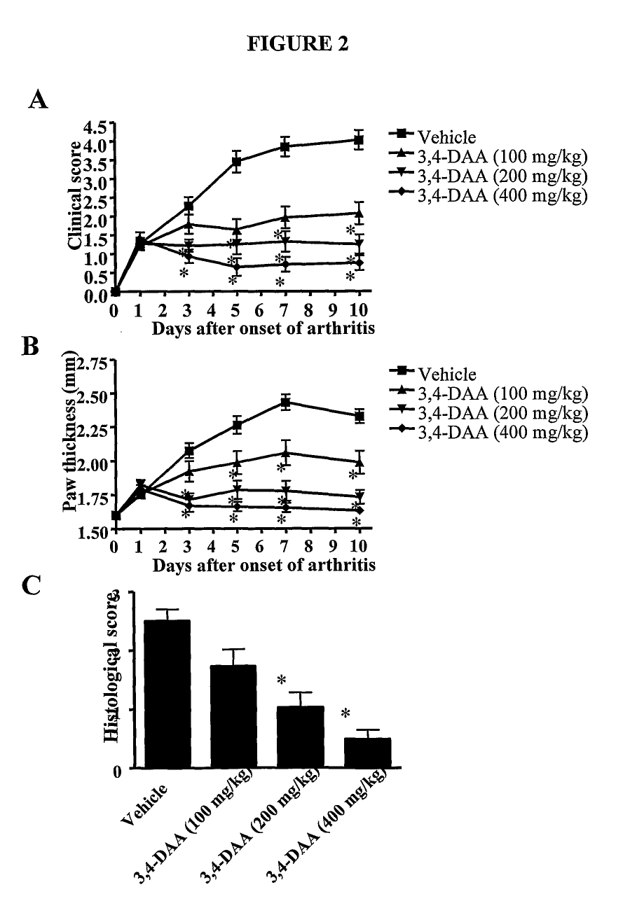

The ability of 3,4-DAA to treat established CIA was tested. Mice were immunised with type II collagen in CFA. On day 1 of clinical arthritis (the day that arthritis was first observed) mice were randomly assigned to different treatment groups and given 3,4-DAA (100 mg/kg/day, 200 mg/kg/day or 400 mg/kg/day) or vehicle alone over a 10 day period. In two separate experiments, a dose-dependent reduction in both clinical scores and paw-swelling was observed in the 3,4-DAA-treated mice (FIG. 1). Significant differences between 3,4-DAA treated and control mice were observed from day 3 until the end of the treatment period (day 10). On day 10 the mice were killed and the first paw to show clinical evidence of arthritis was processed for histology. Joints were examined ‘blindly’ for severity of inflammation and joint erosion. Again, a clear dose-dependent reduction in histological severity of arthritis was observed in the 3,4-DAA-treated mice (FIG. 2).

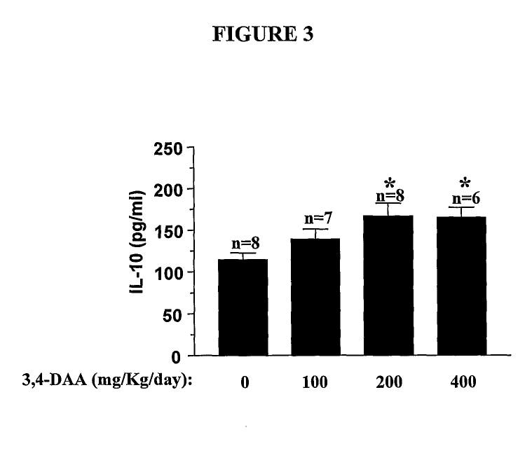

Sera from control and treated mice were analysed for levels of anti-type II collagen IgG1 and IgG2a but no differences were observed between any of the groups. Sera were also analysed for IL-10 production and a dose-dependent increase in circulating IL-10 levels was detected following treatment with 3,4-DAA (FIG. 3).

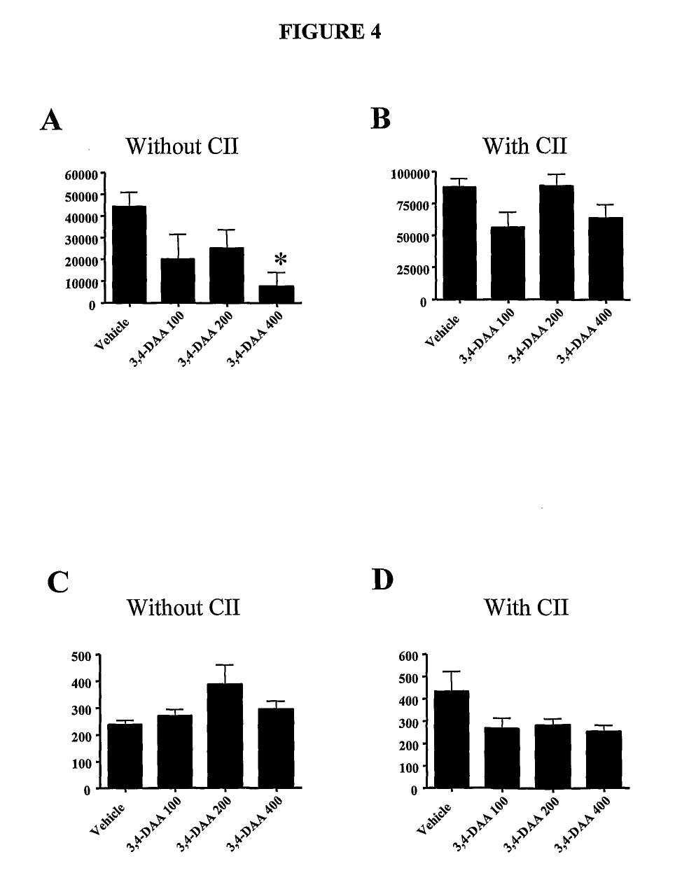

At the end of the experiment draining (inguinal) LNC from control and treated mice were cultured for 72 h in the presence or absence of type II collagen. IFN-γ, IL-5 and IL-10 production was measured by ELISA. IFN-γ production was found to be significantly reduced in the mice given 3,4-DAA at 400 mg/mouse (FIG. 4). However, on re-stimulation with collagen, differences between the groups were not significant, indicating that the ability of the T cells to respond to antigenic stimulation returned to normal once the 3,4-DAA had been removed from the system. IL-5 production was unaffected by treatment with 3,4-DAA, and no IL-10 was detected from any cultures.

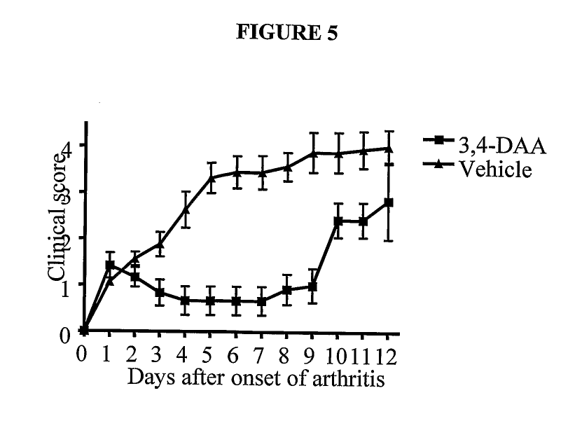

The above data suggests that on removal of 3,4-DAA, LNCs regain the ability to be activated with specific antigen. Therefore, it is clearly of interest to establish what happens in vivo when treatment with 3,4-DAA is stopped. Is there a disease flare and if so, does it occur immediately after cessation of treatment? Hence, a group of arthritic mice were treated from day 1 to day 5 of arthritis with 3,4-DAA (400 mg/kg/day) (FIG. 5). Treatment was then stopped and mice were monitored for a further 7 days. As before, there was a dramatic reduction in arthritis severity during the treatment period. When treatment was stopped on day 5, exacerbation of arthritis was observed from day 9, although the severity of arthritis did not reach that of the control group.

### 3,4-DAA Influences Pain, Reduces Allodynia in Established Arthritis, and Reduces Astrocytic Activation.

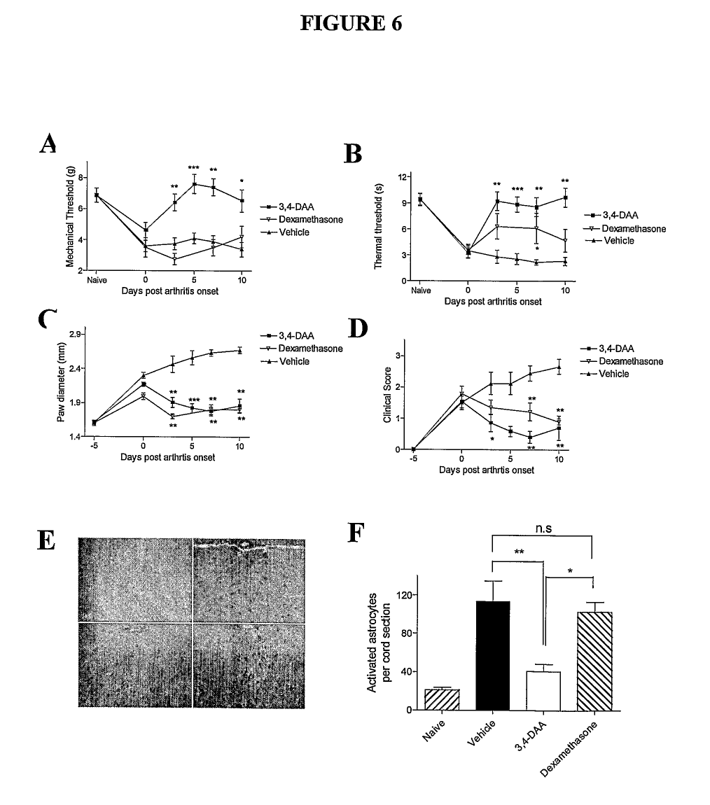

The control of inflammatory pain represents an unmet medical need, and a major challenge for the rheumatologist. The question was therefore addressed of whether 3,4-DAA therapy of established arthritis affected inflammatory pain. Thermal and mechanical allodynia was assessed prior to arthritis onset, on the day of arthritis onset, and up to 10 days following therapy with 3,4-DAA, dexamethasone, or vehicle (FIG. 6). Arthritis induced a 2 and 5-fold decrease in mechanical thresholds on the day of onset, and 5 days post onset respectively (FIG. 6a), and a 3.4-fold decrease in thermal thresholds throughout (FIG. 6b). 3,4-DAA abolished mechanical (FIG. 5a) and thermal allodynia (FIG. 6b) to the levels of non-arthritic animals. In contrast, dexamethasone had only a transient effect on thermal allodynia (FIG. 6b), and no action on mechanical allodynia (FIG. 6a), despite being very effective in controlling inflammation (FIG. 6c,d).

Astrocytic activation has been proposed to be important for the generation of both inflammatory and neuropathic hypersensitivity [Bao et al., 2001, Neuroreport 12:3905-3908; Watkins et al., 2001, Trends Neurosci. 24:450-455]. Astrocytic activation in the spinal cord in CIA was therefore assessed. Upon completion of therapy animals were sacrificed, and GFAP immunohistochemistry was performed in the spinal cord, as a marker of astrocytic activation (FIG. 6e). Quantification of GFAP staining showed that there was a 5.5 fold increase in the number of activated astrocytes in the spinal cord of mice with CIA (FIG. 5f). 3,4-DAA therapy significantly reduced the number of astrocytes detected to a level not significantly different from naïve mice. In contrast, dexamethasone did not affect the level of astrocytic activation.

### 3,4-DAA Inhibits B and T Cell Proliferation In Vitro.

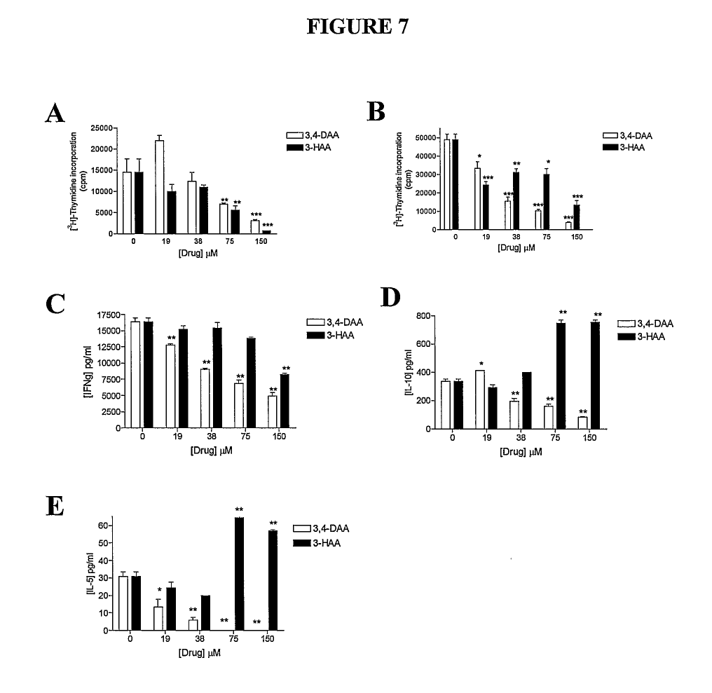

To investigate whether 3,4-DAA has immunomodulatory activity in a manner comparable to its natural analogue, 3-HAA, the anti-proliferative action of 3,4-DAA was compared with 3-HAA on both B and T cells (FIG. 7). Activation of purified B (FIG. 7a) and T cells (FIG. 7b) was induced by anti-CD40, and anti-CD3/CD28 respectively, and proliferation was assessed by ³H-thymidine incorporation. Both 3,4-DAA and 3-HAA dose-dependently inhibited B and T cell proliferation. Inhibition of proliferation was also observed when B cells were stimulated with LPS or anti-IgM. The IC₅₀ for 3,4-DAA, and 3-HAA for inhibition of B cell proliferation was similar; 73 μM and 65 μM respectively. However, the IC₅₀ for inhibition of T cell proliferation was 28 μM for 3,4-DAA, and 100 μM for 3-HAA. In terms of cytokine production, both 3,4-DAA and 3-HAA therapy dose-dependently reduced IFN-γ production by T-cells (FIG. 7c). In contrast 3,4-DAA dose-dependently inhibited IL-10 and IL-5 production (FIGS. 7D, 6E), whilst 3-HAA increased IL-10 and IL-5 production by T-cells. It was concluded that the effects of 3,4-DAA and 3-HAA on T and B cells in vitro were remarkably similar, though not identical. It is of also note that 3-HAA had no action on inflammation or allodynia, when administered therapeutically in CIA at doses of up to 400 mg/kg/day.

### Example 5

### Animal Models of Pain

### Spinal Cord Injury Models

Central pain models are used to test the analgesic effects of flupirtine both with and without morphine. The majority of central pain models are based on spinal cord injury (SCI). Dysesthesia is one of the major life-style altering changes that SCI patients have to cope with. Both spontaneous and evoked pain are frequent sequelae of traumatic or ischemic SCI.

### Neuroma Model

Mice are subjected to complete nerve transection at multiple locations along the sciatic nerve resulting in the development of a neuroma at the proximal nerve stump which consists of regenerative nerve sprouting in all directions. Mice subjected to such surgery typically self attack and mutilate the denervated limb. The mice are then divided into three groups: 1) tranilast; and 2) saline. The animals are then monitored using standard behavioural tests for pain, such as the paw withdrawal threshold or paw flick latency.

### Chronic Constriction Injury Model (CCI or Bennett Model)

Rat have loose ties on the sciatic nerve (left or right side) with four chromic gut ligatures at the mid-thigh level. These rats exhibit behavioural signs of spontaneous pain such as mild to moderate autotomy, guarding, excessive licking and limping of ipsilateral hind paw, and avoidance of placing weight on the injury side. Hyperalgesia due to noxious thermal and mechanical stimuli is detectable, as are cold allodynia and tactile allodynia. All pain signs last for the entire duration of the study (over 2 months). The rats are then divided into three groups: 1) tranilast and 2) saline. The animals are then monitored using standard behavioural tests for pain, such as the paw withdrawal threshold or paw flick latency.

### Partial Sciatic Nerve Ligation Model (PSL or Seltzer Model)

Rats are subjected to ligation of the ipsilateral sciatic nerve at the high thigh level, so that ⅓-½ thickness of the sciatic nerve is trapped in the ligature. Such rats exhibit signs of allodynia to von Frey hair stimulation and hyperalgesia to both thermal and mechno-noxious stimuli with hours of ligation; the symptoms last for over 7 months. Ligated rats also display signs of spontaneous pain in the forms of paw guarding and licking on the injury side. The evoked pain can develop into bilateral patterns. The rats are then divided into three groups: 1) tranilast and 2) saline. The animals are then monitored using standard behavioural tests for pain, such as the paw withdrawal threshold or paw flick latency.

### L5/L6 Spinal Nerve Ligation Model (SNL)

In this model the mice are subjected to unilateral and tight ligation of the L5 and L6 spinal nerve at a location distal to the dorsal route ganglia. Allodynia and hyperalgesia develop quickly after ligation, and last for at least 4 months. Although there are behavioural signs of spontaneous pain (guarding, licking, and lifting of ipsilateral hind paw), autotomy is absent in the SNL. The mice are then divided into three groups: 1) tranilast and 2) saline. The animals are then monitored using standard behavioural tests for pain, such as the paw withdrawal threshold or paw flick latency.

### L5 Spinal Nerve Ligation

Rats are subjected to L5 ligation and exhibit long lasting hyperalgesia and mechanical allodynia. The rats are then divided into three groups: 1) tranilast and 2) saline. The animals are then monitored using standard behavioural tests for pain, such as the paw withdrawal threshold or paw flick latency.

### Sciatic Cryoneurolysis Model (SCN)

Rats are subjected to freezing of the sciatic nerve to produce nerve injury in this model. SCN induces autotomy and touch allodynia which lasts 15 to 21 days. The rats are then divided into three groups: 1) tranilast and 2) saline. The animals are then monitored using standard behavioural tests for pain, such as the paw withdrawal threshold or paw flick latency.

### Inferior Caudal Trunk Resection Model

Rats are subjected to unilateral resection of the inferior caudal trunk between S3 and S4 nerves. Mechanical allodynia and cold or thermal hyperalgesia develop within a day after injury, and can last for weeks. The rats are then divided into three groups: 1) tranilast and 2) saline. The animals are then monitored using standard behavioural tests for pain, such as the paw withdrawal threshold or paw flick latency.

### Sciatic Inflammatory Neuritis Model (SIN)

Rats are injected with zymosan around the sciatic nerve. In this model allodynia is seen hours after the injection. The rats are then divided into three groups: 1) tranilast and 2) saline. The animals are then monitored using standard behavioural tests for pain, such as the paw withdrawal threshold or paw flick latency.

### Cancer Pain Models

Cancer-related pain may be caused by tumor infiltration or compression of nerve, plexus, or roots, immunoreactive and pronociceptive substances released from tumors, or by treatment (chemotherapy, radiation, or surgery).

### Chemotherapy-Induced Peripheral Neuropathy Models

Rats are injected with either vinca alkaloids, platinum compounds or Taxols or other chemotherapeutic agents also capable of inducing neuropathy. The rats are then divided into three groups: 1) tranilast and 2) saline. The animals are then monitored using standard behavioural tests for pain, such as the paw withdrawal threshold or paw flick latency.

### Vincristine-Induced Peripheral Neuropathy Model (VIPN)

Rats are injected daily with vincristine for 10 days (5 consecutive drugs days+2 drug-free days+5 more drug days) resulting in the production of hyperalgesia. The rats are then divided into three groups: 1) tranilast and 2) saline. The animals are then monitored using standard behavioural tests for pain, such as the paw withdrawal threshold or paw flick latency.

Alternatively, rats are subjected to a continuous intravenous vincristine infusion so as to induce in a dose-dependent tactile allodynia. The rats are then divided into three groups: 1) tranilast and 2) saline. The animals are then monitored using standard behavioural tests for pain, such as the paw withdrawal threshold or paw flick latency.

### Taxol-Induced Peripheral Neuropathy Model (TIPN)

Paclitaxel (Taxol) is an antineoplastic agent derived from the Pacific yew tree Taxus brevifolia and is used to treat a variety of cancers, including ovarian and breast tumors, and non-small cell lung cancer. Taxol binds to tubulin (at a site different from that used by the vinca alkaloids) and blocks polymerization of microtubules. Its effectiveness is limited by the development of severe painful peripheral neuropathy that is dose-dependent. The incidence of Taxol neuropathy is estimated to be 50-90%, and is characterised by dysesthesia (e.g. numbness, tingling and burning pain) of the hands and feet. Rats are injected with Taxol resulting in neuropathic pain. The rats are then divided into three groups: 1) tranilast and 2) saline. The animals are then monitored using standard behavioural tests for pain, such as the paw withdrawal threshold or paw flick latency.

### Cisplatin-Induced Peripheral Neuropathy (CIPN)

Cisplatin is used to treat ovarian and small cell lung cancer. Cisplatin induces polyneuropathy that is dose- and treatment duration-dependent, and can last for over 10 years. Rats are subjected to repeated daily injections (i.p.) of cisplatin which produces mechanical allodynia and hyperalgesia. The rats are then divided into three groups: 1) tranilast and 2) saline. The animals are then monitored using standard behavioural tests for pain, such as the paw withdrawal threshold or paw flick latency.

### Cancer Invasion Pain Model (CIP)

Peripheral nerve injury and neuritis models can be used to stimulate peripheral nerve damage due to cancer invasion. Meth A sarcoma cells are implanted around the sciatic in BALB/c mice. There animals develop signs of . . . grows and compresses the nerve. Signs of spontaneous pain (paw lifting) are also visible. The rats are then divided into three groups: 1) tranilast and 2) saline. The animals are then monitored using standard behavioural tests for pain, such as the paw withdrawal threshold or paw flick latency.

### Bone Cancer Pain Models

Bone cancer pain is one of the most common cancer-related pains. Bone cancer can be primary or metastatic from breast, prostate, ovary and lung tumors. Deep pain with a burning and stabbing sensation is often described by bone cancer patents.

### Mouse Femur Bone Cancer Pain Model

Osteolytic mouse sarcoma NCTC2472 cells are injected into the marrow space of the femur bone to induce bone cancer. For histocompatibility, C3H/HeJ mice are used for this model. Within 5 days of sarcoma injection, cancer-induced bone destruction and osteoclastogenesis begin. Signs of spontaneous (nocifensive behaviour, spontaneous flinching) and evoked pain (palpation-evoked flinching), as well as changes in neurochemical markers occur within 14 days, and can be attenuated by osteoprotegerin. The mice are then divided into three groups: 1) tranilast and 2) saline. The animals are then monitored using standard behavioural tests for pain, such as the paw withdrawal threshold or paw flick latency.

### Mouse Calcaneus Bone Cancer Pain (CBC)

NCTC2472 cells are injected into mouse calcaneus bone. Osteolysis, spontaneous pain (paw licking) and evoked pain (mechanical and col allodynia) occur 6 days after implantation and last for at least 16 days. The rats are then divided into three groups: 1) tranilast and 2) saline. The animals are then monitored using standard behavioural tests for pain, such as the paw withdrawal threshold or paw flick latency.

### Rat Tibia Bone Cancer Model (TBC)

MRMT-1 rat mammary gland carcinoma cells are injected into the tibia bone of Sprague-Dawley rats. Bone destruction is detected within 10 days of tumor cell injection. The onset of allodynia and mechanical hyperalgesia are dose (tumor cell number)-dependent, and occur within 10-12 days of tumor cell injection. The rats are then divided into three groups: 1) tranilast and 2) saline. The animals are then monitored using standard behavioural tests for pain, such as the paw withdrawal threshold or paw flick latency.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Bao, L., Zhu, Y., Elhassan, A. M., Wu, Q., Xiao, B., Zhu, J., and Lindgren, J. U. 2001. Adjuvant-induced arthritis: IL-1 beta, IL-6 and TNF-alpha are up-regulated in the spinal cord. Neuroreport 12:3905-3908
Bresalier R. S., Sandler R. S., Quan H., Bolognese J. A., Oxenius B., Horgan K., Lines C., Riddell R., Morton D., Lanas A., Konstam M. A., Baron J. A., (2005) Cardiovascular events associated with rofecoxib in a colorectal adenoma chemoprevention trial. N. Engl. J. Med. 352(11):1092-1102
Drazen J. M., (2005), COX-2 inhibitors—a lesson in unexpected problems. N. Engl. J. Med. 352:1131-1132
Fields H. L., Pain, New York: McGraw-Hill, 1987
FitzGerald, G. A., Coxibs and cardiovascular disease. N. Engl. J. Med. 2004; 351:1709-1711
Haythornthwaite J A, Raja S N, Fisher B, Frank S M, Brendler C B, Shir Y. Pain and quality of life following radical retropubic prostatectomy. J. Urol., 160:1761-4, 1998
Holmes D R Jr, Savage M, LaBlanche J M, Grip L, Serruys P W, Fitzgerald P, Fischman D, Goldberg S, Brinker J A, Zeiher A M, Shapiro L M, Willerson J, Davis B R, Ferguson J J, Popma J, King S B 3rd, Lincoff A M, Tcheng J E, Chan R, Granett J R, Poland M. 2002, Results of Prevention of Restenosis with Tranilast and its Outcomes (PRESTO) trial. Circulation. 106(10):1243-50
Lenzer J., 2005, FDA advisers warn: COX 2 inhibitors increase risk of heart attack and stroke. BMJ. 330(7489):440
Nussmeier N A, Whelton A A, Brown M T, Langford R M, Hoeft A, Parlow J L, Boyce S W, Verburg K M., 2005, Complications of the COX-2 inhibitors parecoxib and valdecoxib after cardiac surgery. N Engl J Med. 352(11): 1081-91
Rainville P, Duncan G H, Price D D, Carrier B, Bushnell M C. Pain affect encoded in human anterior cingulate but not somatosensory cortex. Science 277:968-71, 1997
Solomon S D, McMurray J J, Pfeffer M A, Wittes J, Fowler R, Finn P, Anderson W F, Zauber A, Hawk E, Bertagnolli M; Adenoma Prevention with Celecoxib (APC) Study Investigators., 2005, Cardiovascular risk associated with celecoxib in a clinical trial for colorectal adenoma prevention. N Engl J Med. 352(11): 1071-80
Taenzer P, Melzack R, Jeans M E, Influence of psychological factors on postoperative pain, mood, and analgesic requirements. Pain, 24:331-42, 1986
Watkins, L. R., Milligan, E. D., and Maier, S. F. 2001. Glial activation: a driving force for pathological pain. Trends Neurosci. 24:450-455
Williams, R. O. 2004. Collagen-induced arthritis as a model for rheumatoid arthritis. Methods Mol. Med. 98:207-216

1. A method for inducing analgesia in a subject, said method comprising administering to said subject an effective amount of a compound of formula (I): 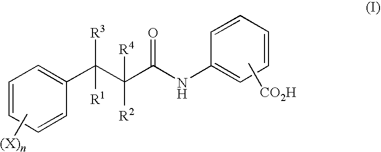
wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

2. A method for prophylactically inducing analgesia in a subject, said method comprising administering to said subject an effective amount of a compound of formula (I): 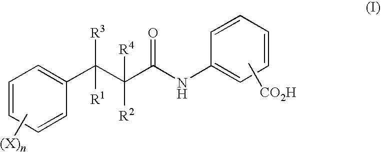
wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄ alkoxy group, or when two X groups are alkyl or alkoxy groups, they may be connected together to form a ring, and n is an integer from 1 to 3.

3. A method for the treatment and/or prophylaxis of a condition in a subject, which condition is characterised by symptoms of pain, said method comprising administering to said subject an effective amount of a compound of formula (I): 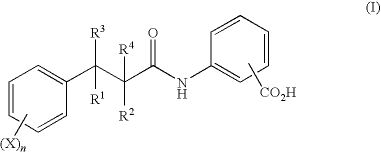
wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, for a time and under conditions sufficient to inhibit or reduce said pain.

4. The method according to claim 1 wherein the carboxyl group is in the 2-, 3- or 4-position of the aromatic ring, at least one of R¹ and R² is a hydrogen atom, R³ and R⁴ taken together form a chemical bond and n is 1 or 2 and each X, which may be the same or different, is selected from halogen, C₁-C₄ alkyl or d-C₄alkoxy.

5. The method of claim 4 wherein the carboxyl group is in the 2-position, both of R¹ and R² are hydrogen atoms and X is selected from halogen and C₁-C₄alkoxy and n is 2 and both X are selected from C₁-C₄alkoxy.

6. The method according to claim 5 wherein said compound is of the formula:

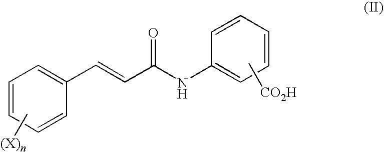

7. The method of claim 6 wherein said compound is selected from the list:
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

8. The method according to claim 7 wherein said compound is 2-[[3-(3,4-dimethoxyphenyl)-1-oxo-2-propenyl]amino]benzoic acid.

9. The method according to claim 1 wherein said pain is inflammatory pain.

10. The method according to claim 9 wherein said inflammatory pain is associated with cancer, infection, inflammation, autoimmune conditions, AIDS, kidney disease, multiple sclerosis, headache, childbirth, menstruation or a post-operative surgical condition.

11. The method according to claim 10 wherein said autoimmune disorder is rheumatoid arthritis, systemic lupus erythematosus or osteoarthritis.

12. The method according to claim 3 wherein said condition is cancer, infection, inflammation, autoimmune conditions, AIDS, kidney disease, multiple sclerosis, headache, childbirth, menstruation or a post-operative surgical condition.

13. A method of downregulating analgesia in a subject, said method comprising administering to said subject an antagonist of a compound of formula (I) or a pharmaceutically acceptable salt thereof.

14. The method according to claim 1 wherein said subject is a mammal.

15. The method according to claim 14 wherein said mammal is a human.

16. A pharmaceutical formulation comprising 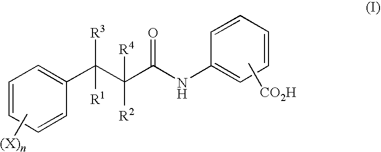
wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3, in a dose effective to induce analgesia in a mammal.

17-20. (canceled)

21. The pharmaceutical formulation of claim 16 wherein said compound is 2-[[3-(3,4-dimethoxyphenyl)-1-oxo-2-propenyl]amino]benzoic acid.

22. The pharmaceutical formulation of claim 16 wherein said pain is inflammatory pain.

23. The pharmaceutical formulation of claim 22 wherein said inflammatory pain is associated with cancer, infection, inflammation, autoimmune conditions, AIDS, kidney disease, multiple sclerosis, headache, childbirth, menstruation or a post-operative surgical condition.

24. The pharmaceutical formulation of claim 23 wherein said autoimmune disorder is rheumatoid arthritis, systemic lupus erythematosus or osteoarthritis.

25-26. (canceled)

27. The pharmaceutical formulation of claim 16 wherein said mammal is a human.
